(19) United States
(12) Patent Application Publication
(10) Pub. No.: US 2025/0127944 A1
(43) Pub. Date: Apr. 24, 2025

(54) HYDROXYL GENERATOR HOUSING WITH MODULAR COMPONENTS

(71) Applicant: The Pyure Company, Boynton Beach, FL (US)

(72) Inventors: Mark MINO, Daytona Beach, FL (US); Jeffrey CHALPAN, Limoges, Ontario (CA); Kelvin MORGAN, West Palm Beach, FL (US)

(21) Appl. No.: 18/491,892

(22) Filed: Oct. 23, 2023

(51) Int. Cl. A61L 2/10; A61L 2/26

(52) U.S. Cl. CPC A61L 2/10; A61L 2/26; A61L 2202/11; A61L 2202/122

(57) ABSTRACT

A hydroxyl generator housing designed for maintainability to allow safe, quick, and easy replacement of hydroxyl generator component parts. This design minimizes repair time in the field for corrective and preventative maintenance, including replacing UV optic lamps, ballast, Programmable Logic Controllers (PLCs), and other electrical components. The housing is formed to include two separate interior regions. The first interior region is for hydroxyl generation/treatment. The second interior region is for a slidable ballast shelf design. The housing design also includes ultraviolet (UV) lamps placed into insertable optics racks. All the hydroxyl generator component parts are accessible through a single cabinet door. Entire component parts can be swapped out in the field, including slidable optics racks holding UV optic lamps, slidable ballast shelves with ballast, PLCs, current sensors, cabling, and other electrical components. The component parts can be returned back to the factory for corrective and preventative maintenance.

### BACKGROUND OF THE INVENTION

Hydroxyls are radicals of the chemical formula ·OH that exist in nature and typically are formed by the interaction of ultraviolet (UV) radiation and atmospheric water vapor:

H₂O+UV radiation→H+·OH

Hydroxyls are very reactive, and therefore are usually short-lived, as they react with compounds around them. This reactivity makes hydroxyls excellent “scavengers” for compounds that act as pollutants, such as bacteria, viruses, mold, odorous compounds, volatile organic compounds (VOCs), poisonous gases, and thousands of other compounds.

In nature, hydroxyls are formed by solar generated UV radiation, and therefore are only formed outdoors. Hydroxyls can also be created indoors by artificially generated UV radiation having wavelengths of 170-270 nm. UV radiation of these wavelengths is also capable of independently decontaminating airstreams of certain particles. Therefore, prior art devices have been constructed which funnel airstreams through banks of UV radiation for the purpose of cleansing the air.

### SUMMARY OF THE INVENTION

Disclosed is a novel hydroxyl generator housing designed for maintainability to allow safe, quick, and easy replacement of hydroxyl generator component parts. This design minimizes repair time in the field for corrective and preventative maintenance, including replacing UV optic lamps, ballast, PLCs, and other electrical components. Further, the design reduces training requirements and reduces specific skill requirements to maintain the system.

The housing is formed to include two separate interior regions. The first interior region is for hydroxyl generation/treatment. The second interior region is for a slidable ballast shelf design. The housing design also includes ultraviolet (UV) lamps placed into insertable optics racks. All the hydroxyl generator component parts are accessible through a single cabinet door.

Entire component parts can be swapped out in the field, including slidable optics racks holding UV optic lamps, slidable ballast shelves with ballast, Programmable Logic Controllers (PLCs), current sensors, cabling, and other electrical components. The component parts can be returned back to the factory for further testing and for corrective and preventative maintenance.

In one example, the hydroxyl generator housing with modular components may include a cuboidal-shaped cabinet formed with six cabinet sides and an interior shelf that separates a first interior region for hydroxyl treatment and a second interior region. The cuboidal-shaped cabinet includes two cabinet sides of the six cabinet sides. The two cabinet sides are parallel and opposite of each other, and each of the two cabinet sides includes at least one opening to provide fluid communication between each of the two cabinet sides and the first interior region for hydroxyl treatment.

The hydroxyl generator housing includes a third cabinet side of the six cabinet sides is perpendicular to the two cabinet sides and includes a plurality of openings into the first interior region and at least one opening to the second interior region for accepting a slidable ballast shelf with electrical ballast disposed thereon for ramping up input voltage high enough to spark two or more ultraviolet (UV) lamps.

The hydroxyl generator housing includes a set of two or more cuboidal-shaped optics racks, each of the cuboidal-shaped optics racks formed with six rack sides defining an optics rack interior space and slidable into the first interior region for hydroxyl treatment through one of the plurality of openings in the third cabinet side so that each of the cuboidal-shaped optics racks is position perpendicular to the slidable ballast shelf. Each of the cuboidal-shaped optics racks includes the two or more UV lamps arranged within the optics rack interior space for allowing air to pass between the openings in each of the two cabinet sides to the first interior region for hydroxyl treatment and through the optics rack interior space. An electrical connector is disposed on an exterior side of one of the six rack sides that is external to the first interior region. The electrical connector provides electrical communication with the set of two or more UV lamps.

The hydroxyl generator housing may include a plurality of electrical components disposed on the third cabinet side to provide access from a single side of the cuboidal-shaped cabinet for each of i) the plurality of electrical components, ii) a removal of the slidable ballast shelf, and iii) a removal of the set of two or more cuboidal-shaped optics racks.

In one example, the electrical components include a programmable logic controller, a computer, or both.

In another example, the hydroxyl generator housing includes a door mechanically attached to the third cabinet side to close over the slidable ballast shelf and the set of two or more cuboidal-shaped optics racks. The door may be formed with a weatherproof seal to provide ingress protection from dust and liquids into the third cabinet side.

The first interior region for hydroxyl treatment and the second interior region of the hydroxyl generator housing may be formed to prevent air from a hydroxyl treatment in the first interior region from passing to the second interior region.

Further, the hydroxyl generator housing may include a blower or fan for moving air from outside the interior region through at least one opening in one of the two cabinet sides into the first interior region for hydroxyl treatment and ejecting air, which has been treated out through the at least one opening in another of the two cabinet sides to outside the interior region.

### BRIEF DESCRIPTION OF THE DRAWINGS

The accompanying figures wherein reference numerals refer to identical or functionally similar elements throughout the separate views, and which together with the detailed description below are incorporated in and form part of the specification, serve to further illustrate various embodiments and to explain various principles and advantages all in accordance with the present invention, in which:

FIG. 1 is a first perspective view of a hydroxyl generator, with a door open to show various electrical components and a set of optics racks, according to an example of the invention;

FIG. 2 is a first-side view of a hydroxyl generator of FIG. 1, according to an example of the invention;

FIG. 3 is a second perspective view of a hydroxyl generator of FIG. 2, to show the overlap of ultra violet (UV) lamps in two optical racks, various electrical components and a set of optics racks, according to an example of the invention;

FIG. 4 is a second-side view of a hydroxyl generator of FIG. 1, to show various electrical components, a slidable ballast shelf, and a set of optics racks, according to an example of the invention;

FIG. 5 is a third perspective view of a hydroxyl generator of FIG. 1, to show the overlap of the UV lamps and with a door open, according to an example of the invention;

FIG. 6 is an orthographic projection of a hydroxyl generator with a translucent cabinet of FIG. 1, to show various electrical components, a slidable ballast shelf, and a set of optics racks, according to an example of the invention;

FIG. 7 is a top view of an optics rack, according to an example of the invention; and

FIG. 8 is a perspective explosion view of an optics rack of FIG. 7, according to an example of the invention.

### DETAILED DESCRIPTION OF THE PREFERRED EMBODIMENTS

Prior art devices deployed in certain applications, such as food and beverage spaces, are periodically disinfected along with all surfaces to meet sanitary requirements. Disinfection is typically done using chemical cleaners, and water is sprayed over exposed surfaces.

Further, prior art devices are often deployed in applications such as on a cruise ship, in which equipment space is very limited, providing very limited access for maintenance.

The present invention provides a hydroxyl generator that is designed for easy maintenance even in tight spaces. The present invention provides ingress protection against dust and water in applications where this is required. The hydroxyl generator generates hydroxyls and secondary oxidants, which are, and whose effects are, relatively long-lived, and which can be used to purify large volumes of air and objects that come into contact with that air.

The present invention also provides a method of disinfecting and/or deodorizing air, which comprises the steps of generating hydroxyls using the hydroxyl generator of the invention and then distributing the hydroxyls over the space to be disinfected and/or deodorized using air moving equipment, such as fans or blowers. For example, the air around smelly commercial facilities, such as pig farms, and rendering plants can be deodorized using the hydroxyl generators according to the invention so that air exiting from the facility is less odorous or even odor-free.

Another use of the hydroxyl generator according to the invention is in the deodorizing of indoor air spaces following fires, floods, or other odor-generating activities. A further use of the hydroxyl generator according to the invention is the decontamination and/or remediation of biological pathogens, such as Covid-19, including biological weapons, such as anthrax.

### Non-Limiting Terminology

The terms “a” or “an,” as used herein, are defined as one or more than one. The term plurality, as used herein, is defined as two or more than two. The term another, as used herein, is defined as at least a second or more.

The term “and” in the phrase “one of A, B, and C” means either A or B or C or any combination of A, B, and C.

The term “coupled” or “coupling” as used herein, is defined as “connected” although not necessarily directly and not necessarily mechanically. Coupled can be sewn using stitching of various forms, glue, welding, heat sealing, stapling, other mechanical, physical or chemical means.

The terms “including” and “having” as used herein are defined as comprising (i.e., open language).

The term “substantially” means to a large degree but not absolute.

### Overview of Hydroxyl Generator Housing

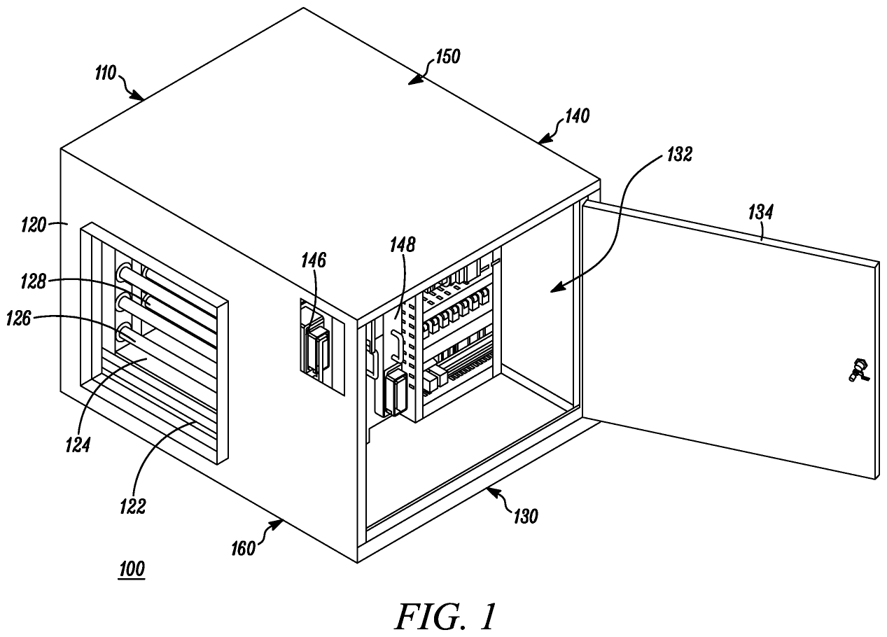

FIG. 1 is a first perspective view of a hydroxyl generator 100. A hydroxyl generator 100 is formed in a cuboidal-shaped cabinet formed with six cabinet sides 110, 120, 130, 140, 150, 160, as shown. All six cabinet sides are formed with solid walls typically constructed from metal, such as aluminum, but other materials or combinations of materials, such as can be used to construct the hydroxyl generator and its components. For example, materials such as metals, alloys, composites, plastics, ceramics, and other inorganic or organic materials or combinations thereof may be used. Preferably, the interior surface of the housing is reflective of UV radiation. In one example, the interior surface of the housing of the housing is made of aluminum, more preferably unpolished aluminum. The cabinet need not be made of a single piece of material but may be composed of two or more pieces which fit closely together so as to define an interior space. The cabinet may be fabricated using stamping, molding, and bending. One or more of the sides may be held together by welds, fasteners, or adhesives (not shown).

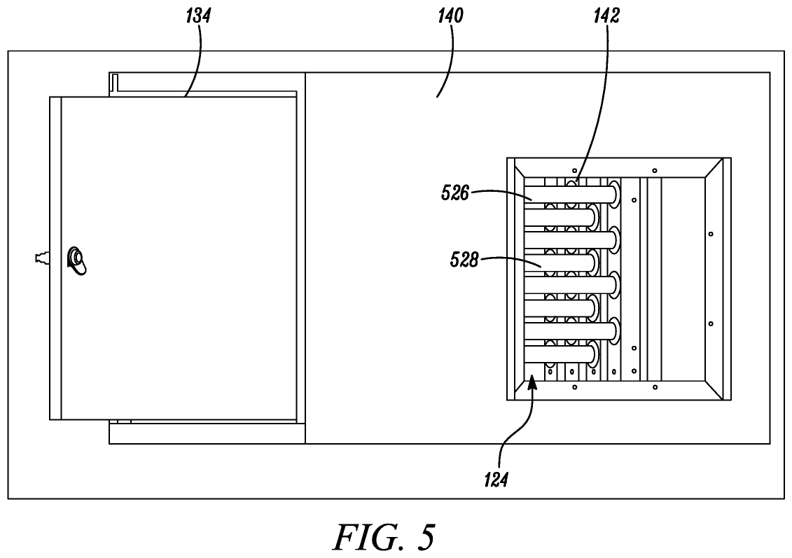

The cuboidal-shaped cabinet includes two cabinet sides 120, 140 in parallel and opposite of each other, as shown. The two cabinet sides 120, 140 include at least one opening 122, 142 (note opening 142 is shown in FIG. 5 below) to provide fluid communication between each of the two cabinet sides and a first interior region 124 for hydroxyl treatment. The openings 122 and 142 are formed to be rectangular to fit with rectangular duct size of 16 inches by 16 inches but other rectangular duct sizes are within the true scope of the present invention.

Cabinet side 120 with opening 122 depicts a UV lamps 126, 128 as part of the set of optics racks 146, 148. The UV lamps 126, 128 are positioned inside the first region 124 for hydroxyl treatment.

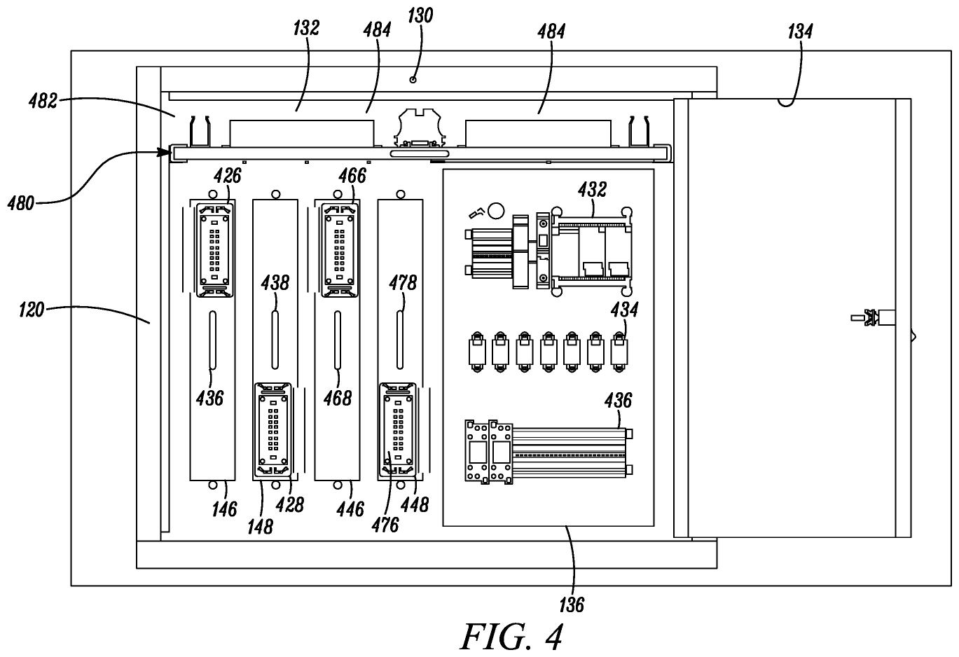

A third cabinet side 130 is perpendicular to the two cabinet sides 120, 140 as shown. The third cabinet side 130 includes a plurality of openings 132 into the first interior region 124. The third cabinet side 130 includes at least one opening to a second interior region 482 for accepting an interior slidable ballast shelf 480 (note that shelf 480, and the second interior region 482 are depicted in FIG. 4 below). Typically, the first interior region 124 for hydroxyl treatment and the second interior region 482 are sealed from each other. This sealing prevents air undergoing a hydroxyl treatment in the first interior region 124 from passing to the second interior region 482.

In this example, a door 134 is opened to show various electrical components 136 and the set of optics racks 146, 148.

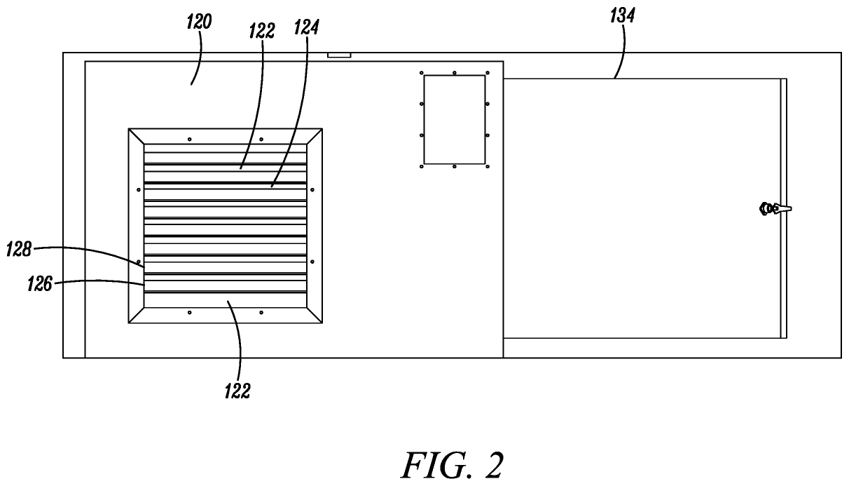

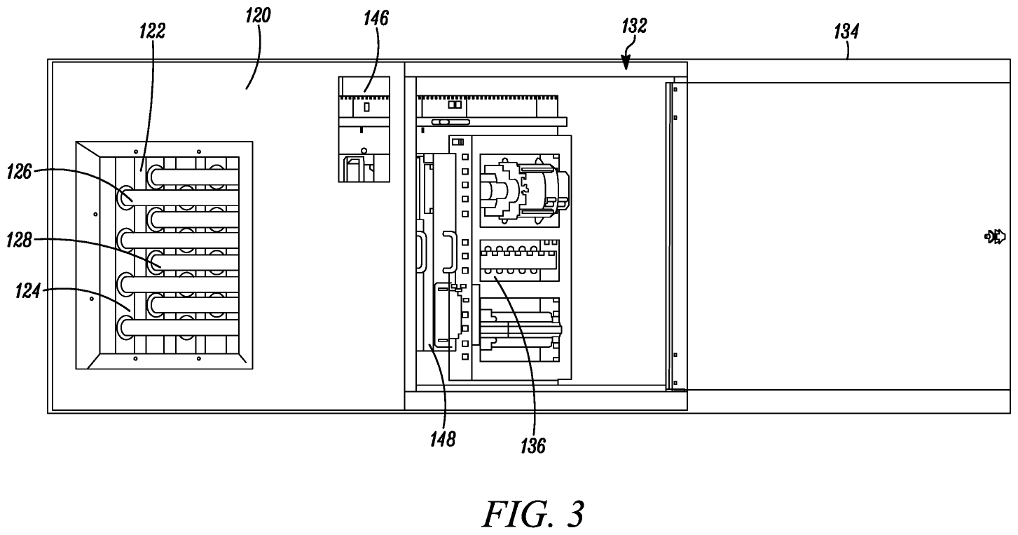

Continuing further FIG. 2 is a first side view of a hydroxyl generator 100 of FIG. 1. FIG. 3 is a second perspective view of a hydroxyl generator of FIG. 2, to show the overlap of the UV lamps in the set of optics racks 146, 148, and with a door open to show various electrical components 136 and a set of optics racks 146, 148.

### Single Side Access to Hydroxyl Generator Housing

FIG. 4 is a second-side view 130 of a hydroxyl generator 100 of FIG. 1. The door 134 is open to illustrate at least one opening to the second interior region 482 for accepting a slidable ballast shelf 480 for ultraviolet (UV) lamps 126, 128 mounted on optical racks 126, 128. Also, UV lamps (not shown) mounted in optical racks 446, 448.

The door 134 in an open position provides access to various electrical components 136 and a slidable ballast shelf 480. In one example the, door 134 in a closed position includes a weatherproof seal to provide ingress protection from dust and liquids into opening 132. Unlike prior art hydroxyl generators, the present invention provides single-side access to maintain and replace any electrical components, including a plurality of cuboidal-shaped optical racks 146, 148, 446, 448 and electrical components from a single-side. Each of the optical racks 146, 148, 446, 448 includes an electrical connectors 426, 428, 466, 476 that are electrically coupled through cables (not shown for simplicity) to the UV optic lamps 126, 128. Further in this example, each of the optical racks 146, 148, 446, 448 includes a handle 436, 438, 468, and 478. The handles make it easier to slide each optical rack in and out of the first interior region 124 for hydroxyl treatment. When the optical racks are inserted into position, they serves to define a portion of the housing of the hydroxyl generator.

The slidable ballast shelf 480 includes electrical ballast 484. The ballast 484 ramps an input voltage required to spark the UV lamps 126, 128. The ballast 484 are in electrical communication with a cables (not shown for simplicity) that attaches to the ballast shelf 480 and one of the electrical connectors 426, 428, 466, 476. Once the UV lamps 126, 128 are sparked and an arc is achieved, the ballast 484 limits the voltage to a lower working voltage to keep the lamp illuminated while regulating the power draw of the UV lamps 126, 128.

The various electrical components 136 shown in FIG. 4 include a programmable logic controller (PLC) 432. In one example, a computer may be used in place of or in conjunction with the PLC 432. Also shown in FIG. 4 is a set of current sensors 434 for monitoring the current delivered to each of the UV lamps. A wiring terminal block 436 secures two or more wires together from the PLC 432, current sensors 434, UV lamps 126, 128, and other electronics, including resettable circuit breakers.

FIG. 5 is a third perspective view of a hydroxyl generator of FIG. 1 with door 134 opened to show the overlap of the UV lamps 526, 528 located in optical racks 446, 448 within the first interior region 124 for hydroxyl treatment.

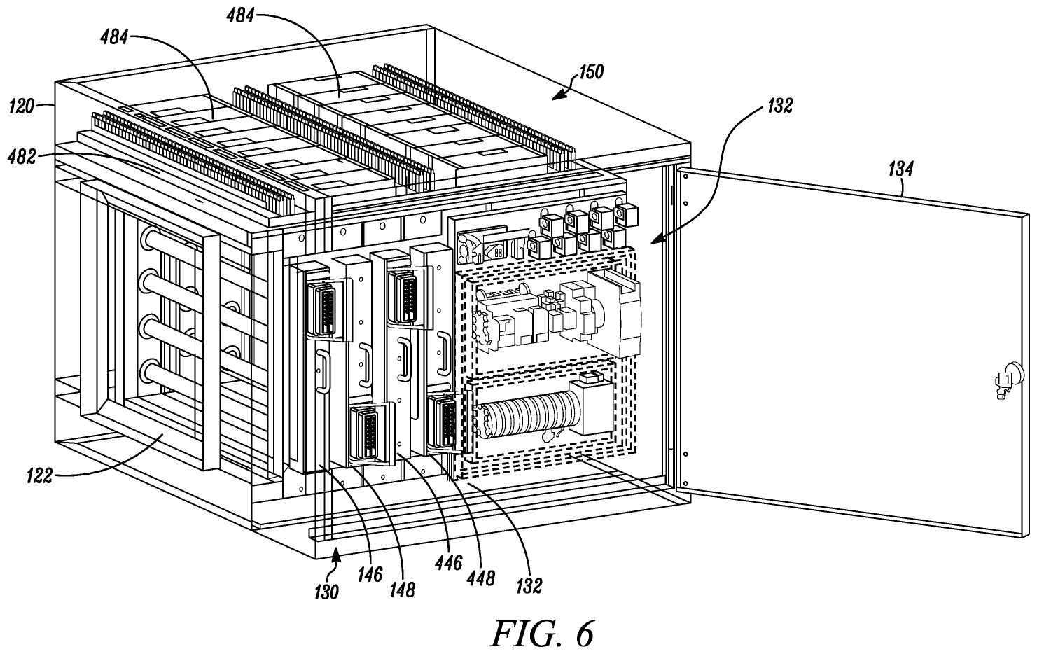

FIG. 6 is an orthographic projection of a hydroxyl generator with a translucent cabinet of FIG. 1, with a door 134 open to show various electrical components 136, a slidable ballast shelf 480, and a set of optics racks 146, 148, 446, 448 as described above.

### Optics Rack

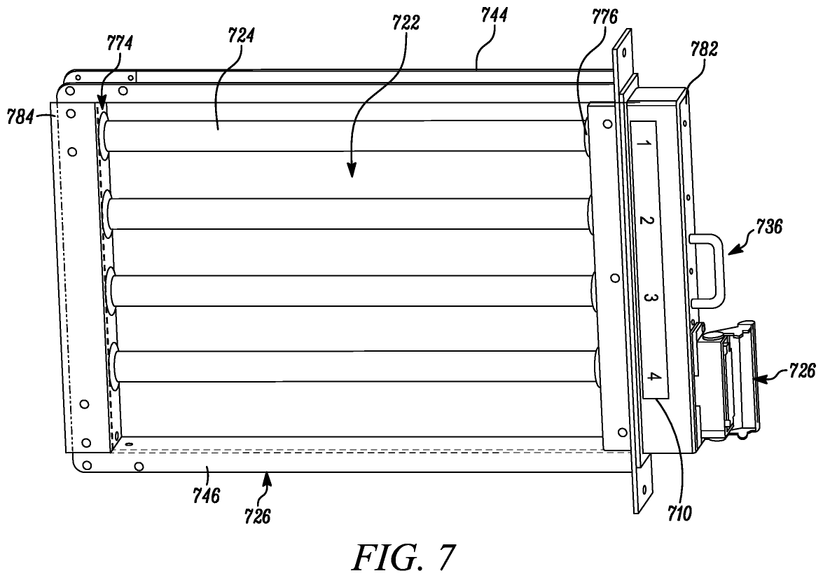

FIG. 7 is a top view of an optics rack 726. The optics rack 726 depicted may be one of the set of optics racks 146, 148, 446, 448 described above. The optics rack, as shown, is cuboidal-shaped optics rack formed with six sides 744, 746, 782, 784, and two open sides that allow air to pass over one or more UV lamps 724 in an optics rack interior space 722. The relative position of each of these UV lamps is important and is described in the commonly owned U.S. Pat. Nos. 9,168,323, 8,765,072, and 8,257,649, the entire disclosure of each is hereby incorporated by reference in its entirety.

Preferably, the UV lamps 724 are positioned generally in a parallel arrangement within rows of optic racks 146, 148, 446, 448. Each row is slightly offset from the immediately adjacent rows, as may be seen in FIGS. 2, 3, and 5, when adjacent racks are flipped 180 degrees relative to each other. See FIG. 4. This design of the flipping the adjacent optics rack reduces the number of distinct field replaceable parts needed to be stocked. The desired offset of UV lamps between rows is maintained as further described below). Further, in this example, the UV lamps 724 are referenced by a numbered position 710 to assist with diagnosing, maintenance, and replacement. Each UV Lamp is held in a firm position within the optical rack 726 with mounts 774 and 776, as shown.

The housing need not be made of a single piece of material but may be composed of two or more pieces that fit closely together so as to define an optics rack interior space 722. When the removable racks are inserted into position, they serve to define the housing.

Preferably, the interior surface of the housing is reflective of UV radiation. In one preferred embodiment, the interior surface of the housing is made of aluminum, more preferably unpolished aluminum.

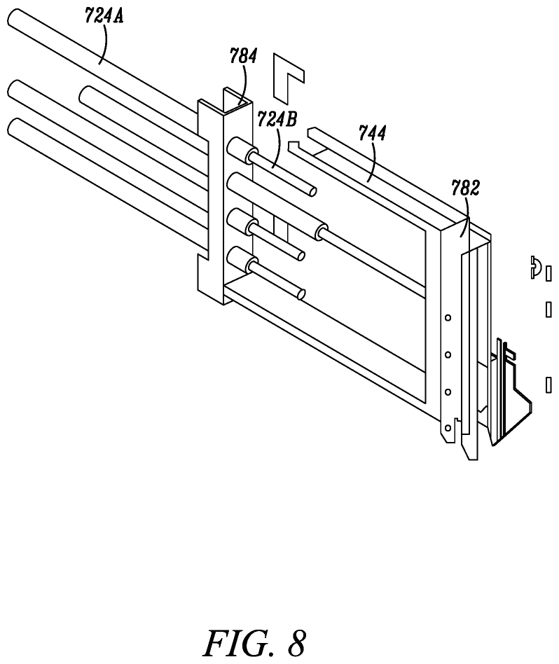

FIG. 8 is a perspective explosion view of an optics rack of FIG. 7. In this view the sides 744, 746, 782, 784, and the UV lamps 724 are shown in a disassembled state. Note a protective sleeve 724A surrounds the UV lamp 724B. The protective sleeve 724A should be transparent to the UV radiation generated by the lamp. A preferred sleeve is made of a material known as double virgin quartz.

### Ultra Violet Optic Lamps

The UV radiation used to generate the hydroxyls comes from UV optic lamps. These UV optic lamps are available commercially. Preferably, at least some of the UV lamps used in this hydroxyl generator of the invention provide UV radiation having a multiple wavelengths for treatment of air inside the hydroxyl generator as well in the efficient generation of hydroxyls, but at least some of the UV lamps should generate UV radiation centered around the wavelength of about 185 nm. It is also preferred that the UV lamps be surrounded by a protective sleeve. The protective sleeve should be transparent to the UV radiation generated by the lamp. A preferred sleeve is made of a material known as double virgin quartz.

In many applications, the UV lamp will have a linear shape, but, as will be discussed in more detail below, it can also be bent or curved.

Alternatively, a single UV lamp can be used inside the housing, either a straight, curved, or bent lamp, which is positioned inside a housing so that its corona fills substantially all the interior space of that housing.

The UV lamps are positioned such that when air passes through the first interior region 124 in which the UV lamps are operating, superior hydroxyls are generated.

Air can be fed through the hydroxyl generator by conventional air moving devices (not shown in the drawings), such as, for example, but not limited to, fans, pumps, blowers, etc. Baffles and other physical structures may also be used to regulate how long it takes air to pass through the housing. Preferably, air will be present inside the housing for a period of between 150 to 500 milliseconds.

In a preferred embodiment, each UV lamp is cylindrical and has an exterior diameter of between ¼ to 2 inches, and is surrounded by a cylindrical sleeve with an interior diameter of ½ to 2¼ inch and an exterior diameter of ¾ to 2½ inches. In the preferred embodiment, the UV lamp produces a corona with a diameter of 2¾ to 4 inches, and the lamps are positioned such that their centers are 2 to 4 inches apart and ¾ to 2 inches from the inside surface of the housing. Preferably, the UV lamps are positioned in rows, with each row being slightly offset from the immediately adjacent rows, as may be seen in FIGS. 2, 3, and 5.

### Non-Limiting Examples

The present invention has been shown and described herein in what is considered to be the most practical and preferred embodiment. It is recognized, however, that departures may be made therefrom within the scope of the invention and that obvious modifications will occur to a person skilled in the art.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed as the best mode contemplated for carrying out this invention, but that the invention will include all embodiments falling within the scope of the appended claims.

We claim:

1. A hydroxyl generator housing with modular components comprising:
a cuboidal-shaped cabinet formed with six cabinet sides and an interior shelf that separates a first interior region for hydroxyl treatment and a second interior region, the cuboidal-shaped cabinet including
two cabinet sides of the six cabinet sides, the two cabinet sides are parallel and opposite of each other, and each of the two cabinet sides includes at least one opening to provide fluid communication between each of the two cabinet sides and the first interior region for hydroxyl treatment;
a third cabinet side of the six cabinet sides, the third cabinet side is perpendicular to the two cabinet sides and includes a plurality of openings into the first interior region, and at least one opening to the second interior region for accepting a slidable ballast shelf with electrical ballast disposed thereon for ramping up input voltage high enough to spark two or more ultraviolet (UV) lamps;
a set of two or more cuboidal-shaped optics racks, each of the cuboidal-shaped optics racks formed with six rack sides defining an optics rack interior space and slidable into the first interior region for hydroxyl treatment through one of the plurality of openings in the third cabinet side so that each of the cuboidal-shaped optics racks is position perpendicular to the slidable ballast shelf, each of the cuboidal-shaped optics racks including
the two or more UV lamps arranged within the optics rack interior space for allowing air to pass between the openings in each of the two cabinet sides to the first interior region for hydroxyl treatment and through the optics rack interior space; and
an electrical connector disposed on an exterior side of one of the six rack sides that is external to the first interior region, the electrical connector provides electrical communication with the set of two or more UV lamps.

2. The hydroxyl generator housing of claim 1, further comprises:
a plurality of electrical components disposed on the third cabinet side to provide access from a single side of the cuboidal-shaped cabinet for each of i) the plurality of electrical components, ii) a removal of the slidable ballast shelf, and iii) a removal of the set of two or more cuboidal-shaped optics racks.

3. The hydroxyl generator housing of claim 2, wherein the electrical components include a programmable logic controller, a computer, or both.

4. The hydroxyl generator housing of claim 1, further comprises:
a door mechanically attached to the third cabinet side to close over the slidable ballast shelf and the set of two or more cuboidal-shaped optics racks.

5. The hydroxyl generator housing of claim 4, wherein the door is formed with a weatherproof seal to provide ingress protection from dust and liquids into the third cabinet side.

6. The hydroxyl generator housing of claim 1, wherein the first interior region for hydroxyl treatment and the second interior region are formed to prevent air from a hydroxyl treatment in the first interior region from passing to the second interior region.

7. The hydroxyl generator housing of claim 1, wherein the two or more UV lamps are in a parallel arrangement to each other and arranged within the optics rack interior space, the two or more UV lamps, when generating UV radiation, create in proximity to a surface of each of the two or more UV lamps a near-infrared (NIR) region, wherein the NIR regions of adjacent UV lamps overlap of the two or more UV lamps each other by less than 25% of a radius of the NIR regions.

8. The hydroxyl generator housing of claim 7, wherein a first of the cuboidal-shaped optics rack of the set of two or more cuboidal-shaped optics racks is positioned 180 degrees relative to a second of cuboidal-shaped optics rack in the set of two or more cuboidal-shaped optics racks when slidably inserted into the first interior region for hydroxyl treatment through one of the plurality of openings in the third cabinet side, the NIR regions of adjacent UV lamps on each of the set of two or more cuboidal shaped optics rack overlap each other by less than 25% of a radius of the NIR regions.

9. The hydroxyl generator housing of claim 1, further comprising:
a blower or fan for moving air from outside the interior region through the at least one opening in one of the two cabinet sides into the first interior region for hydroxyl treatment and ejecting air which has been treated out through the at least one opening in another of the two cabinet sides to outside the interior region.
